(19) United States
(12) Patent Application Publication
(10) Pub. No.: US 2013/0220318 A1
(43) Pub. Date: Aug. 29, 2013

(54) CERAMIC ROTARY VALVE FOR AN ANESTHETIC VAPORIZER

(71) Applicant: GE Medical Systems Global Technology Company, LLC, (US)

(72) Inventors: Hongkui GU, Shanghai (CN); Bin Tan, Shanghai (CN)

(73) Assignee: GE MEDICAL SYSTEMS GLOBAL TECHNOLOGY COMPANY, LLC, Waukesha, WA (US)

(21) Appl. No.: 13/781,190

(22) Filed: Feb. 28, 2013

(30) Foreign Application Priority Data
Feb. 28, 2012 (CN) 201210059823.2

(51) Int. Cl. A61M 16/01

(52) U.S. Cl. CPC A61M 16/01
USPC 128/203.12; 251/304

(57) ABSTRACT

Embodiments of the present invention relate to a ceramic rotary valve for an anesthetic vaporizer, the valve body of the rotary valve comprising a ceramic material. Embodiments of the present invention also provide an anesthetic vaporizer comprising the ceramic rotary valve and an anesthesia machine comprising the anesthetic vaporizer. Furthermore, embodiments of the present invention also provide a method of utilizing an anesthetic vaporizer comprising a ceramic rotary valve to control anesthetics concentration output by the anesthetic vaporizer.

### BACKGROUND OF THE INVENTION

Embodiments of the present invention relate to an anesthetic vaporizer, and more particular, to a rotary valve for the anesthetic vaporizer.

An anesthesia machine is a medical apparatus commonly used in clinical operation, which is used for delivering anesthetics into a patient body via mechanical circuit and finally implementing anesthesia on the patient. An anesthesia machine mainly comprises such components as a gas supply and delivery system, an anesthetic vaporizer, a respirator, a monitoring and controlling system, and so forth. As a key component of the anesthesia machine, the vaporizer is used for transforming anesthetics into vapor, and utilizing a certain amount of carrier gas to obtain anesthetic gas with a certain concentration. A valve (for example, a rotary valve) is typically used in the vaporizer to control the concentration of anesthetics output therefrom.

A structural illustration of an anesthetic vaporizer is shown in FIG. 1. As illustrated in FIG. 1, fresh gas enters into the vaporizer from the inlet of the vaporizer, and is then split and directed into two different airways, wherein in one airway, the first portion of the fresh gas is directly outputted after being adjusted by a thermostat, this airway being called “the bypass”; and in the other airway, the remaining fresh gas enters into the vaporizing chamber and mixed with anesthetics. Finally, these two fresh gas streams will be proportionally mixed again to be the final output.

The rotary valve plays a key role in the output concentration performance. When the rotary valve is rotated to the desired concentration scale, the gas output from the vaporizing chamber will go through the corresponding allopelagic spiral groove on the bottom of the rotary valve, and finally becomes an acceptable output. Thus, to obtain a desired precise concentration, as a critical dimension of the rotary valve, the depth dimension of the groove must be preferentially guaranteed.

Since the rotary valve is a moving component, thousands of rotations will lead to surface wear and will directly affect the depth dimension of the spiral groove, which will eventually influence the output concentration.

Thus, it is desirable to improve wear resistance of the rotary valve with the processing quality guaranteed.

U.S. Pat. No. 4,059,657 describes an anesthetic vaporizer having a flow calibration control valve, which is used for proportionally distributing inlet gas between the vaporizing chamber and the main bypass channel, to obtain the desired anesthetics concentration. A rotary multi-ports valve having a plurality of parallel laminar-flow channels is operated in discrete steps in order to add anesthetic vapor from the anesthetic chamber to a preset stream of inlet gas in a predictably increasing manner, so as to proportionally distribute the gas. An auxiliary gas bypass for temperature compensation has a taper valve, which has a limited opening controlled by a motor corresponding to the temperature in the vaporizing chamber.

U.S. Pat. No. 3,575,168 describes an anesthetic vaporizing equipment comprising a rotary proportional control plate valve used for controlling two separated streams of inlet gas flow, one of which passes through liquid volatile anesthetics to form vapor, while the other is bypassed through a tube controlled by the temperature compensation bypass valve, wherein this plate valve also controls an outflow of the gas-vapor mixture, and the bypass valve has a non-constant resistance property.

However, the anesthetic vaporizing equipments described in these patents and the rotary valves used therein do not appropriately address the problem of the wearing of the valve surfaces.

Therefore, it is desirable to provide a rotary valve in this art capable of keeping surfaces thereof substantially unworn and keeping dimensions substantially unaffected after experiencing a large amount of rotations.

### BRIEF DESCRIPTION OF THE INVENTION

Embodiments of the present invention provide a rotary valve for an anesthetic vaporizer comprising a valve body, the valve body made of or comprising a ceramic material.

Embodiments of the present invention also provide a method for controlling anesthetics concentration output from an anesthetic vaporizer, wherein the vaporizer comprises a first airway and a second airway arranged in parallel, wherein the first airway is arranged with a vaporizing chamber and a rotary valve, the rotary valve being downstream of the vaporizing chamber. The method comprises flowing a fresh gas into the anesthetic vaporizer, and splitting the fresh gas into a vaporizing chamber stream and a bypass stream, wherein the vaporizing chamber stream and the bypass stream enter into the first airway and the second airway, respectively. The method further comprises mixing the vaporizing chamber stream with anesthetics in the vaporizing chamber; flowing the resulting mixture through the valve body of the rotary valve made of ceramic material; rotating the rotary valve to a desirable concentration scale; mixing the mixture from the first airway through the rotary valve with the bypass stream flowing through the second airway; and outputting the mixing product from the anesthetic vaporizer.

Embodiments of the present invention further provide an anesthetic vaporizer comprising a rotary valve. The rotary valve further comprises a valve body made of or comprising a ceramic material.

Embodiments of the present invention further provide an anesthesia machine comprising a vaporizer. The vaporizer comprises a rotary valve that comprises a valve body made of or comprising a ceramic material.

### BRIEF DESCRIPTION OF THE DRAWINGS

Other features and advantages of the present invention will be apparent from the following detailed description of the preferred embodiments taken in conjunction with accompanying drawings, which illustrate embodiments of the invention by way of example.

FIG. 1 illustrates an anesthetic vaporizer; and

FIG. 2 illustrates a valve body of a rotary valve for the anesthetic vaporizer in FIG. 1 according to an embodiment of the invention.

The accompanying drawings are used only to illustrate embodiments of the invention, but not to limit the scope thereof.

### DETAILED DESCRIPTION OF THE INVENTION

An embodiment of the present invention is now described in detail, an example of which is shown in the accompanying drawings. Reference numerals are used in the detailed description to indicate features in the drawings. Like numerals are used in the drawings and description to represent like features and components.

The following example is provided as an illustration of an embodiment of the invention, rather than a limitation of the invention. In fact, those skilled in the art will understand that modifications and alterations may be made to embodiments of the invention without departing from the scope and spirit of the invention. For example, a feature shown or described as a portion of an embodiment can be used in another embodiment, so as to yield yet another embodiment. Thus, embodiments of the present invention are intended to cover such modifications and alterations that come within the scopes of the appended claims and their equivalents.

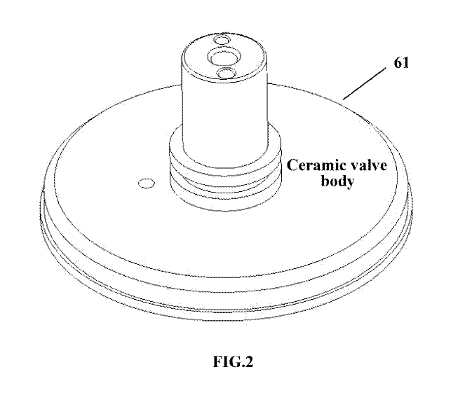

FIG. 2 illustrates an embodiment of a valve body 61 of a rotary valve 6 for an anesthetic vaporizer. The valve body 61 may comprise a spiral groove (not shown) on its bottom, by which the flow rate of the gas through the rotary valve 6 may be adjusted.

In an embodiment of the present invention, the valve body 61 is made of or at least partially comprises a ceramic, so that the wear resistance of the rotary valve 6 can be greatly improved. Due to the rotary valve 6, especially with the enhancement of the valve body 61, the dimensions of the spiral groove in the valve body can be guaranteed, whereas in the cases of similar valve bodies made of conventional materials, the dimensions thereof, especially the depth dimensions, will be affected by the wear of the valve body.

The ceramic material of the valve body 61 may be zirconia, alumina, carborundum, silicon nitride, or any combination thereof. However, it should be understood that, these materials are only exemplary embodiments which can be used as ceramic material of the valve body 61, and any material having desired wear resistance, chemical and mechanical stability, and biocompatibility is suitable for the valve body 61.

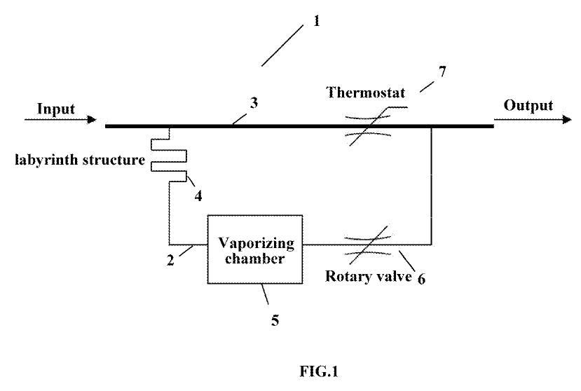

As shown in FIG. 1, the rotary valve 6 illustrated in FIG. 2 which comprises the valve body 61 comprising a ceramic material, can be used for the anesthetic vaporizer 1. The anesthetic vaporizer 1 comprises an inlet, an outlet, a first airway 2 and a second airway 3 arranged in parallel. In an embodiment, the anesthetic vaporizer 1 comprises a labyrinth structure 4, a vaporizing chamber 5 and a rotary valve 6 on the first airway 2. The labyrinth structure 4 is arranged between the inlet and the vaporizing chamber 5, while the rotary valve 6 is arranged downstream of the vaporizing chamber 5, wherein in operation, a first portion of the fresh gas flowing into the inlet passes into the first airway 2, through the labyrinth structure 4, and into the vaporizing chamber 5 where it is mixed with anesthetics. The resulting mixture then flows through the rotary valve 6. In an embodiment, a thermostat 7 may be arranged in the second airway 3, wherein in operation, a second portion of the fresh gas flowing into the inlet passes into the second airway 3 and through the thermostat 7, then is proportionally mixed again with the mixture from the rotary valve 6 in the first airway 2, and finally outputs from the outlet.

Of course, it should be understood that the structure of the above anesthetic vaporizer 1 is not intended to be used as a limitation. The rotary valve 6 having the valve body 61 of ceramic material can be refitted in any suitable existing vaporizer of the prior art.

By utilizing the rotary valve 6 in the vaporizer comprising a valve body 61, which comprises or is comprised of a ceramic material, the output concentration can be more accurate and stable during a long-term use of the vaporizer. At the same time, by solving the wear problem for the valve of the vaporizer, the working life of the vaporizer can be prolonged. Consequently, it will come true that no concentration calibration is required.

Furthermore, in addition to ensuring the accuracy and stability of the output concentration, the properties of non-toxic, good chemical stability, and biocompatibility of the ceramic material applied to the valve body will guarantee the safety of patients, and high mechanical stability will avoid stress deformation in the underside of the rotary valve.

The rotary valve shown in FIG. 2 comprising the valve body 61, which comprises or is comprised of a ceramic material, can be used for a method of controlling the concentration of anesthetic(s) through the rotary valve. The method comprises flowing a fresh gas into the anesthetic vaporizer 1, and splitting it into a vaporizing chamber stream and a bypass stream, wherein the vaporizing chamber stream and the bypass stream enter into the first airway 2 and the second airway 3, respectively; and mixing the vaporizing chamber stream with anesthetics in the vaporizing chamber 5, and flowing the resulting mixture through the valve body 61 of the rotary valve 6 comprising ceramic material. The method further comprises rotating the rotary valve 6 to a desirable concentration scale; and mixing the mixture from the first airway 2 through the rotary valve 6 with the bypass stream flowing through the second airway 3, and outputting it from the anesthetic vaporizer.

According to an embodiment, the method further comprises flowing the vaporizing chamber stream through the labyrinth structure 4 in the first airway 2, prior to entering into the vaporizing chamber 5.

According to another embodiment, the method further comprises arranging a thermostat 7 in the second airway 3 to adjust the flow rate of the bypass stream.

Although the invention is described only in the embodiments illustrated in FIGS. 1 and 2, those skilled in the art should understand that many modifications and alterations which conform to the spirit of the invention may be made without departing from the protection scope defined in the claims.

In an embodiment, the ceramic material may be selected from a group consisting of zirconia, alumina, carborundum, and silicon nitride.

In another embodiment, the valve body of the rotary valve may comprise a spiral groove on its bottom.

In another embodiment, the rotary valve adjusts a flow rate of the gas flowing through its spiral groove by rotation.

An embodiment of the present invention also provides an anesthesia machine comprising an anesthetic vaporizer as described above.

In a further embodiment of the present invention, an anesthetic vaporizer comprising a rotary valve is provided where the rotary valve comprises a valve body made of ceramic material. The anesthetic vaporizer further comprises an inlet, an outlet, a first airway and a second airway arranged in parallel, a labyrinth structure, a vaporizing chamber, and a thermostat. The labyrinth structure, the vaporizing chamber and the rotary valve are arranged in the first airway with the labyrinth structure arranged between the inlet and the vaporizing chamber, while the rotary valve is arranged downstream of the vaporizing chamber. The thermostat is arranged in the second airway. The anesthetic vaporizer is configured so that, in operation: (i) a first portion of a fresh gas flowing into the inlet passes into the first airway, through the labyrinth structure, and into the vaporizing chamber, where it is mixed with anesthetics, the resulting mixture flows through the rotary valve; and (ii) a second portion of the fresh gas flowing into the inlet passes into the second airway, through the thermostat, and then mixes proportionally again with the mixture from the rotary valve in the first airway, and finally outputs from the outlet.

In yet another embodiment of the present invention, an anesthesia machine is provided. The anesthesia machine comprises a vaporizer comprising a rotary valve comprising a valve body made of ceramic material. The vaporizer further comprises an inlet, an outlet, a first airway and a second airway arranged in parallel, a labyrinth structure, a vaporizing chamber, and a thermostat. The labyrinth structure, the vaporizing chamber and the rotary valve are arranged in the first airway with the labyrinth structure arranged between the inlet and the vaporizing chamber, while the rotary valve is arranged downstream of the vaporizing chamber. The thermostat is arranged in the second airway. The anesthetic vaporizer is configured so that, in operation: (i) a first portion of a fresh gas flowing into the inlet passes into the first airway, through the labyrinth structure, and into the vaporizing chamber, where it is mixed with anesthetics, the resulting mixture flows through the rotary valve; and (ii) a second portion of the fresh gas flowing into the inlet passes into the second airway, through the thermostat, and then mixes proportionally again with the mixture from the rotary valve in the first airway, and finally outputs from the outlet.

Furthermore, the aforementioned method may also comprise flowing the vaporizing chamber stream through the labyrinth structure in the first airway prior to entering the vaporizing chamber. The method may even further comprise arranging a thermostat in the second airway to adjust the flow rate of the bypass stream.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A rotary valve for an anesthetic vaporizer comprising:
a valve body comprising a ceramic material.

2. The rotary valve of claim 1, wherein the ceramic material is zirconia, alumina, carborundum, silicon nitride, or any combination thereof.

3. The rotary valve of claim 1, wherein the valve body of the rotary valve comprises a top and a bottom, the bottom comprising a spiral groove.

4. The rotary valve of claim 3, wherein the rotary valve adjusts a flow rate of a gas flowing through the spiral groove by rotation.

5. An anesthetic vaporizer comprising:
a rotary valve comprising a valve body made of ceramic material.

6. The anesthetic vaporizer of claim 5, further comprising:
an inlet;
an outlet;
a first airway and a second airway arranged in parallel;
a labyrinth structure;
a vaporizing chamber; and
a thermostat arranged in the second airway;
wherein the labyrinth structure, the vaporizing chamber and the rotary valve are arranged on the first airway, with the labyrinth structure arranged between the inlet and the vaporizing chamber, and the rotary valve arranged downstream of the vaporizing chamber; and
wherein the anesthetic vaporizer is configured such that that, in operation, (i) a first portion of a fresh gas flowing into the inlet passes into the first airway, through the labyrinth structure, and into the vaporizing chamber where it is mixed with anesthetics, and the resulting mixture flows through the rotary valve; and (ii) a second portion of the fresh gas flowing into the inlet passes into the second airway and through the thermostat, and then mixes proportionally again with the mixture from the rotary valve in the first airway, and finally outputs from the outlet.

7. A method of controlling anesthetics concentration output from an anesthetic vaporizer, wherein the vaporizer comprises a first airway and a second airway arranged in parallel, wherein the first airway is arranged with a vaporizing chamber and a rotary valve, the rotary valve downstream of the vaporizing chamber, the method comprising:
flowing a fresh gas into the anesthetic vaporizer, and splitting the fresh gas into a vaporizing chamber stream and a bypass stream, wherein the vaporizing chamber stream and the bypass stream enter into the first airway and the second airway, respectively;
mixing the vaporizing chamber stream with anesthetics in the vaporizing chamber, and flowing the resulting mixture through the valve body of the rotary valve comprising a ceramic material;
rotating the rotary valve to a desirable concentration scale; and
mixing the mixture from the first airway through the rotary valve with the bypass stream flowing through the second airway, and outputting the mixing product from the anesthetic vaporizer.

8. The method of claim 7, further comprising:
flowing the vaporizing chamber stream through the labyrinth structure in the first airway prior to entering into the vaporizing chamber.

9. The method of claim 7, further comprising:
arranging a thermostat in the second airway to adjust the flow rate of the bypass stream.

10. An anesthesia machine comprising:
a vaporizer, the vaporizer comprising a rotary valve comprising a valve body made of ceramic material.

11. The anesthesia machine of claim 10, wherein the vaporizer further comprises:
an inlet;
an outlet;
a first airway and a second airway arranged in parallel;
a labyrinth structure;
a vaporizing chamber; and
a thermostat arranged in the second airway;
wherein the labyrinth structure, the vaporizing chamber and the rotary valve are arranged on the first airway, with the labyrinth structure arranged between the inlet and the vaporizing chamber, and the rotary valve arranged downstream of the vaporizing chamber; and
wherein the anesthetic vaporizer is configured such that that, in operation, (i) a first portion of a fresh gas flowing into the inlet passes into the first airway, through the labyrinth structure, and into the vaporizing chamber where it is mixed with anesthetics, and the resulting mixture flows through the rotary valve; and (ii) a second portion of the fresh gas flowing into the inlet passes into the second airway and through the thermostat, and then mixes proportionally again with the mixture from the rotary valve in the first airway, and finally outputs from the outlet.
